(19) United States
(12) Patent Application Publication
(10) Pub. No.: US 2002/0095120 A1
(43) Pub. Date: Jul. 18, 2002

(54) Automatic injection device

(76) Inventors: Andre Larsen, Dragor (DK); Gabriel Jorgensen, Copenhagen K (DK); Kim Steengaard, Birkerod (DK); Peter Christian Klitgaard, Smorum (DK); Jens Moller Jensen, Copenhagen K (DK)

Correspondence Address: PATENT DEPARTMENT, SKADDEN, ARPS, SLATE, MEAGHER & FLOM LLP, FOUR TIMES SQUARE, NEW YORK, NY, 10036, US

(21) Appl. No.: 09/942,526

(22) Filed: Aug. 29, 2001

(30) Foreign Application Priority Data
Aug. 29, 2000 (DK) PA 2000 01274

(51) Int. Cl. A61M 5/00

(52) U.S. Cl. 604/187

(57) ABSTRACT

The invention relates to an automatic injection device, which automatically injects a pre-measured quantity of fluid medicine when a tension spring is released. The tension spring moves the ampoule and the injection needle from a storage position to a deployed position when it is released; the content of the ampoule is thereafter expelled by the tension spring forcing a piston forward inside the ampoule. After the fluid medicine has been injected, torsion stored in the tension spring is released and the injection needle is automatically retracted back to its original storage position.

The invention relates to an injection device for automatically injecting a pre-measured quantity of medicine or other fluent material into a mammal body.

It is sometimes necessary to administer a single pre-measured quantity of medicine to a person. It may be diabetics or allergy suffers who may require an emergency injection of glycogen or anti-histamines, or other patients who require only one pre-measured dose of medicine.

Such an automatic emergency injection device must be able to administer the pre-measured quantity in three steps after actuating the device. First the needle itself must be moved from a hidden storage position to a deployed position where it penetrates the skin of the patient. It is both from a security point of view and from a psychological point of view important that the needle is kept out of the patients sight at all times during the injection. Secondly the pre-measured dose must be injected, without the injection starting before the needle is properly deployed into the skin of the patient. Finally the used needle must be retracted in to the housing to prevent any risk of accidental needle stick injury and to prevent any unnecessary stress on the patient.

EP 516.473 discloses one type of automatically operated injection device, which is able to inject a pre-measured quantity in the three steps mentioned above. The device comprises of two coiled springs. When the release button is activated, the first spring first causes the ampoule-holder carrying the ampoule to move forward, and the needle carried on the ampoule to penetrate the skin of the patient. The first spring then urged the piston rod carrying the piston further forward as the fluid is expelled from the ampoule. When the piston has travelled a predetermined distance and expelled a predetermined amount of the content of the ampoule the second spring retracts the ampoule-holder thereby retracting the ampoule and the needle into the housing.

It is an object of the present invention to provide an automatic injection device, which is simple in construction and reliable in use, having only a few movable parts thereby minimising the risk of malfunction.

This is obtained by an automatic injection device comprising:

a housing,

an ampoule holder including an ampoule, said ampoule holder being slidably mounted in said housing,

a piston slidably mounted within said ampoule, a resilient element for causing said ampoule holder first to slide with respect to said housing from a storage position to a deployed position, secondly causing said piston to slide within said ampoule from a first position to a second position such that a predetermined amount of the content of said ampoule are expelled,

means for retracting said ampoule holder to said storage position when said piston has reached said second position, and

means for releasable retaining said resilient element in an armed position, which injection device according to the invention is characterised in that, said resilient element causes said ampoule holder to retract to said storage position.

When a retainer has been released the resilient element first pulls a nut member towards the distal end of the device. The nut member is through a thread connection connected to a piston rod. Since both the piston rod and the nut member is locked against rotation when moved forward, it shoots the piston rod forward towards the distal end. This movement first moves an ampoule holder from a storage position to a deployed position where a needle penetrates into the skin of the patient. The ampoule holder then abuts an internal stop in the device which initialised the second movement where the piston rod and the piston is moved forward within the ampoule expelling a predetermined part of the content of the ampoule out through the needle. Finally the resilient element operates the nut member and the piston rod in such a way that the ampoule holder and the needle is being retracted into the injection device. After use the injection device is disposed of.

In a first embodiment of the injection device according to the invention the resilient element is a single tension spring having a distal end and a proximal end, which tension spring is coiled between the distal and the proximal end. In this way it is ensured that the nut member is pulled in the distal direction inside the housing when the spring is released from the armed position.

When, as disclosed in claim 3, the tension spring is located in the proximal part of the housing opposite the ampoule, it is possible to keep the ampoule visible for inspection. By making the front part of the housing and/or the ampoule holder transparent or partly transparent it will be possible for the user of the injection device to visible inspect the content of the ampoule without having the vision obstructed by the presence of a spring or other mechanical object.

In another embodiment of the injection device according to the invention one end of the tension spring is fasten to the housing and the other end is fasten to a movable nut member. Between these two fastening points the tension spring is winded for performing torsion on the movable nut member. This makes the nut member movable in the longitudinal direction inside the housing, and forces at the same time the nut member to rotate.

The nut member is though, according to yet another embodiment of the injection device according to the invention, locked against rotation due to a number of nut guiding tracks provided in the housing and by which tracks the movable nut member is guided. In this way it is ensured that the nut member can only rotate when it is free of contact with the guiding tracks.

When, as disclosed in claim 6, the movable nut member, after having travelled a predetermined distance is set free to rotate due to the torsion of the winded tension spring, it is ensured that the piston rod is hoisted backwards inside the housing.

The longitudinal length of the nut guide tracks formed in the housing according to yet another embodiment of the injection device according to the invention, determines the predetermined distance the movable member has to travel. The distance the nut member has to travel in the tracks before it reached the rotation chamber at the end of the guiding tracks and is set free to rotate determines the size of the dose being released from the ampoule when the device is being used. By changing the length of the tracks it is possible to make syringe with different predetermined doses.

When as disclosed in claim 8, the piston rod abutting the piston is coupled to the ampoule holder by a one way coupling only allowing the piston rod to move forward relative to the ampoule holder, it is ensured that the piston rod connected to the one way coupling can only move in the forward direction relatively to the ampoule and the ampoule holder.

According to yet another embodiment of the injection device according to the invention the piston rod abutting the piston is provided with saw-teeth which co-operates with saw-teeth on the one way coupling or on the ampoule holder. Due to this one way coupling it is ensured that the ampoule holder will be pulled backwards whenever the piston rod is pulled backwards in the housing.

According to the last embodiment of the injection device according to the invention the releasable retainer is released using a opener preferably provided in a cap located at the distal end of said housing. In this way it is ensured that the cap has been removed before the device is actuated.

The invention will be explained more fully below in connection with a preferred embodiment and with references to the drawings in which:

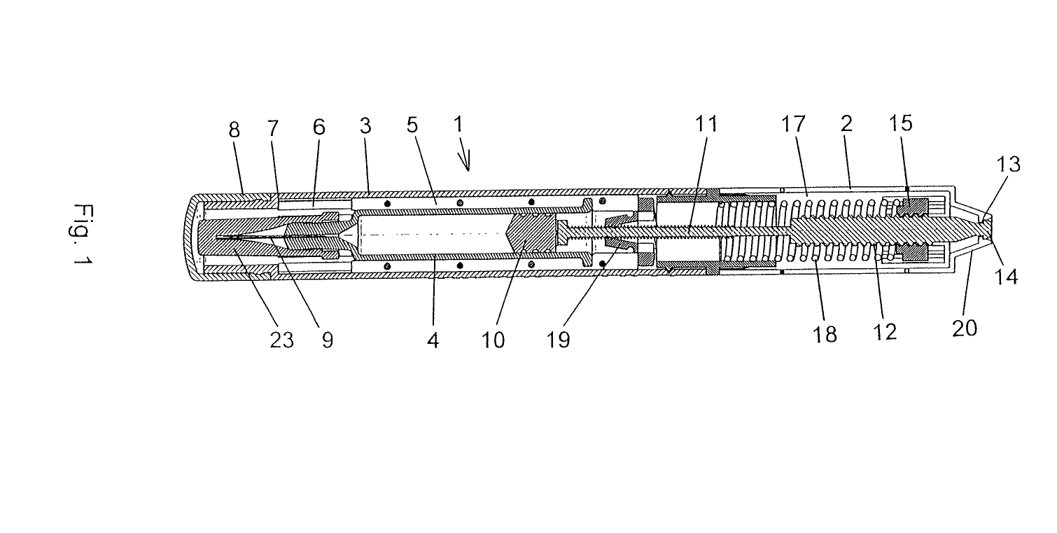

FIG. 1 Shows a sectional view of the automatic syringe according to the invention, which syringe is shown in the armed position.

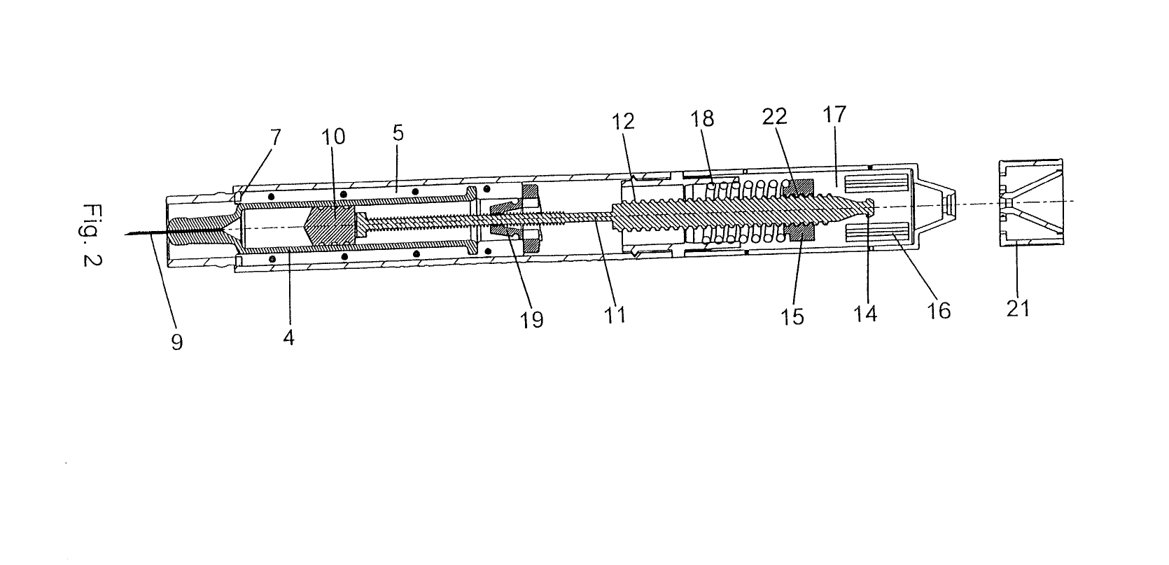

FIG. 2 Shows a sectional view of the automatic syringe according to the invention, which syringe is shown in the deployed position with part of the content expelled

The figures are schematic and simplified for clarity, and they just show details, which are essential to the understanding of the invention, while other details are left out. Through-out, the same reference numerals are used for identical or corresponding parts.

Initially it may be convenient to define that, the term “distal end” of the housing 1 is meant to refer to the end carrying the injection needle 9, whereas the term “proximal end” is meant to refer to the opposite end carrying the retainer mechanism.

FIG. 1 and 2 shows an automatic injection device, which is particularly adapted for the self-administration of medicines or other fluent materials. The syringe comprises an elongated housing 1, which is divided into a first part 2 and a second part 3. The first part 2 forming the proximal end of the syringe has incorporated a retaining mechanism 13, 14, 20 for retaining the piston rod 11 in the armed position. The second part 3 forming the distal end of the syringe is used to cover up the needle and carries at its distal end a cap 8.

In the syringe a ready for use ampoule 4 is mounted in a ampoule holder 5. The ampoule 4 is at the distal end provided with a needle 9 to be inserted into the skin of a patient. The needle 9 is covered by a needle cover 23, which needle cover 23 can be connected to the cap 8 in such a way that the needle cover 23 is removed together with the cap 8 prior to injection.

The ampoule holder 5 is free to slide within the second part 3 of the housing 1, but only in the longitudinal direction due to the presence of a number of guiding tracks 6 located on the interior surface of the second part 3 of the house 1. These guiding tracks 6 leads into an internal stop 7 for the ampoule-holder 5 also provided on the interior surface of the housing 1. In that way limiting the distance the ampoule-holder 5 can travel forward within the second part 3 of the housing 1.

The piston rod 11 is inserted in the ampoule 4 and abuts a piston 10 placed inside the ampoule 4. At the opposite end the piston rod 11 is provided with a contraction 13 and a retainer 14 formed as a stud. The retainer 14 is retained by a number of resilient arms 20, which arms 20 are formed as a part of the first part 2 of the housing 1. When the resilient arms 20 are moved away from each other the retainer 14 is able to slip through the hole formed between the resilient arms 20. To move the resilient arms 20 away from each other an opener 21 can be used. The opener 21 has a cone-shaped central part, which, when inserted over the retainer 14, moves the resilient arms 20 away from each other, and thereby allows the retainer 14 to slip trough the hole formed between the resilient arms 20. The opener 21 can be formed as an integral part of the cap 8.

The piston rod 11 is coupled to the ampoule holder 5 through a one way coupling 19 which can be formed as a part of the ampoule-holder 5 or as a separate piece connected to the ampoule-holder 5 e.g. by a number of screws. The one way coupling 19 is provided with saw-teeth, which co-operates with saw-teeth on the piston rod 11. Due to these saw-teeth the piston rod 11 can only move forward relative to the one way coupling 19 and to the ampoule holder 5. If the piston rod 11 is pulled backwards inside the housing 1 it will bring the ampoule-holder 5 with it backwards.

The one way coupling 19 is formed in a way making the piston rod 11 non rotational relative to the one way coupling 19 and to the ampoule holder 5 e.g. by making the cross section of the piston rod 11 non circular. The ampoule holder 5 is due to the guiding tracks 6 non-rotational mounted in the housing 1.

The piston rod 11 is near the proximal end of the housing 1 provided with a helical thread 12, on which thread 12 a movable nut member 15 having a corresponding internal thread 22 is placed. This nut member 15 is on its distal end connected to a tension spring 18 which spring 18 has its distal end opposite the nut member 15 connected to the housing 1 approximately where the first part 2 and the second part 3 of the housing is connected to each other.

The tension spring 18 is stretched in such a way that it, when released from its armed position, moves the nut member 15 towards the distal end of the housing 1. The first part of the housing 2 is on the inside provided with nut guide tracks 16. The nut member 15 slides in these tracks 16 and is thereby locked against rotation. The tracks 16 lead into a rotation chamber 17 located within the first part 2 of the housing 1, in which chamber 17 the nut member 15 is free to rotate. The forward movement of the nut member 15 continues until the nut member 15 is moved free of the nut guide track 16 and into the rotation chamber 17. The tension spring 18 is winded for performing torsion on the movable nut member 15, as will be explained later.

To operate the automatic injection device the cap 8 is first removed and preferably placed on the proximal end of the housing 1 to disarm the injection device. The syringe is then placed with the most distal end placed against the skin on the person to be injected.

When the retainer 14 is released as shown on FIG. 2 the tension spring 18 pulls the nut member 15 towards the distal end of the housing 1. Since the nut member 15 is locked against rotation it shoots the piston rod 11 towards the distal end of the housing 1. Due to the reluctance in the one way coupling and the presence of fluid in the ampoule 4, the ampoule 4 and the ampoule holder 5 is first moved forward together with the piston rod 11 from a storage position to a deployed position where the needle penetrates into the skin of the patient. When the distal end of the ampoule holder 5 abuts the internal stop 7 in the guiding track 6, as shown in FIG. 2, the ampoule holder 5 is prevented from moving further forward in the longitudinal direction. The tension spring 18 however keeps moving the nut member 15 forward thereby moving the piston rod 11 and the piston 10 forward within the ampoule 4 and thereby expelling a part of, or the whole content of, the ampoule 4 out through the needle 9.

When the nut member 15 is moved out of engagement with the nut guide track 16 and the spring 18 is fully contracted the forward movement of the piston rod 11 and the piston 10 is stopped. In that way the amount of medicine to be expelled from the ampoule is determined by the stroke of the piston, which again is determined by the length of the nut guide tracks 16 in combination with the characteristics of the spring 18.

A variety of syringes expelling different doses of pre-measured quantity of medicine can very easy be manufactured simply by making the nut guide tracks 16 of different length. Another way of making such a variety would be to make different nut members 15, where only a part of the nut member 15 is guided in the nut guide track 16.

As shown in FIG. 2 the nut member 15 will start to rotate once it enters the rotational chamber 17, due to the torsional impact of the winded tension spring 18. As the nut member 15 is prevented from moving further forward, and at the same time forced to rotate, the piston rod 11 will be hoisted up through the nut member 15 due to the thread on the piston rod 11 and the corresponding internal thread 22 on the nut member 15..When the piston rod 11 is being moved in the proximal direction within the housing 1 it will pull the one way coupling 19 and the ampoule holder 5 towards the proximal end of the housing 1, and in that way retract the used needle 9 into the second part 3 of the housing 1.

Some preferred embodiments have been shown in the foregoing, but it should be stressed that the invention is not limited to these, but may be embodied in other ways within the subject matter defined in the following claims.

Listing of parts

1. Housing

2. First part, of housing

3. Second part, of housing

4. Ampoule

5. Ampoule holder

6. Guiding tracks

7. Internal stop

8. Cap

9. Needle

10. Piston

11. Piston rod

12. Piston rod thread

13. Piston rod contraction

14. Retainer

15. Nut member

16. Nut guide tracks

17. Rotation chamber

18. Tension spring

19. One way coupling

20. Resilient arms

21. Opener

22. Internal thread, of nut member

23. Needle cover

1. An automatic injection device comprising:
a housing,
an ampoule holder including an ampoule said ampoule holder being slidably mounted in said housing,
a piston slidably mounted within said ampoule,
a resilient element for causing said ampoule holder first to slide with respect to said housing from a storage position to a deployed position, secondly causing said piston to slide within said ampoule from a first position to a second position such that a predetermined amount of the content of said ampoule are expelled,
means for retracting said ampoule holder to said storage position when said piston has reached said second position, and
means for releasable retaining said resilient element in an armed position
characterised in that
said resilient element causes said ampoule holder to retract to said storage position.

2. An automatic injection device according to claim 1, characterised in that said resilient element is a tension spring having a distal end and a proximal end, said tension spring being coiled between said distal and said proximal end.

3. An automatic injection device according to claim 2, characterised in that said tension spring in the longitudinal direction is located in the proximal part of said housing opposite said ampoule.

4. An automatic injection device according to claim 2 or 3, characterised in that said tension spring has said distal end fasten in said housing and said proximal end fasten to a movable nut member, said tension spring being winded between said two fastenings points for performing torsion on said movable nut member.

5. An automatic injection device according to claim 4, characterised in that said movable nut member is locked against rotation due to a number of nut guiding tracks provided in said housing and by which tracks said movable nut member is guided.

6. An automatic injection device according to claim 5, characterised in that said movable nut member after having travelled a predetermined distance is set free to rotate due to the torsion of said winded tension spring.

7. An automatic injection device according to claim 6, characterised in that said predetermined distance is determined by the longitudinal length of said nut guiding tracks formed in said housing.

8. An automatic injection device according to anyone of the preceding claims, characterised in that a piston rod abutting said piston is coupled to said ampoule holder by a one way coupling only allowing said piston rod to move forward relative to said ampoule holder.

9. An automatic injection device according to claim 8, characterised in that said piston rod abutting said piston is provided with saw-teeth which co-operates with saw-teeth on said one way coupling or on said ampoule holder.

10. An automatic injection device according to anyone of the claims, characterised in that said releasable retainer is released using an opener preferably provided in a cap located at the distal end of said housing.
